(19) United States
(12) Patent Application Publication
(10) Pub. No.: US 2020/0054755 A1
(43) Pub. Date: Feb. 20, 2020

(54) ROS-DEGRADEABLE HYDROGELS

(71) Applicant: VANDERBILT UNIVERSITY, Nashville, TN (US)

(72) Inventors: Craig L. Duvall, Nashville, TN (US); Mukesh K Gupta, Nashville, TN (US); John R. Martin, Nashville, TN (US); Bryan R. Dollinger, Nashville, TN (US)

(21) Appl. No.: 16/547,348

(22) Filed: Aug. 21, 2019

Related U.S. Application Data
(63) Continuation of application No. 15/132,076, filed on Apr. 18, 2016, now abandoned.
(60) Provisional application No. 62/149,294, filed on Apr. 17, 2015.

(51) Int. Cl. A61K 47/32; C08G 75/08; C08F 283/00; C08F 287/00; A61K 9/00; A61K 41/00; A61K 9/06

(52) U.S. Cl. CPC A61K 47/32; C08G 75/08; C08F 283/00; A61K 9/06; A61K 9/0019; A61K 41/0028; C08F 287/00

(57) ABSTRACT

The presently-disclosed subject matter includes a polymer (i.e., copolymer) comprising a thermally responsive block and a hydrophobic block. In some embodiments the copolymer is a terpolymer. Specific embodiments include a thermo-responsive, ROS degradable ABC triblock terpolymer comprising poly(propylenesulfide)-block-poly(N,N-dimethylacrylamide)-block-poly(N-isopropylacrylamide) (PPS-b-PDMA-b-PNIPAAM).

### RELATED APPLICATIONS

This application is a continuation of U.S. patent application Ser. No. 15/132,076, filed Apr. 18, 2016, which claims the benefit of U.S. Provisional Application Ser. No. 62/149,294, filed Apr. 17, 2015, the entire disclosures of which are incorporated herein by this reference.

### TECHNICAL FIELD

The presently-disclosed subject matter relates to hydrogels that are degradable by reactive oxygen species (ROS). In particular, the presently-disclosed subject matter relates to ROS-degradable, thermoresponsive hydrogels and methods for delivering an active agent using the same.

### BACKGROUND

Injectable, in situ forming biodegradable polymeric hydrogels that are responsive to environmental or externally-applied stimuli (such as temperature, pH, ultrasonic sound, light, or ionic strength) represent promising platforms for encapsulation and delivery of drugs and/or cells in a variety of biomedical applications. Thermo-responsive hydrogels based on poly(N-isopropylacrylamide) (PNIPAAM) have been studied for drug and cell delivery applications because of PNIPAAM's lower critical solution temperature (LCST) of about 32° C., which is close to body temperature and enables injection of solutions at ambient temperature that gel in situ at physiologic temperature. This overcomes practical manufacturing and storage issues related to pre-fabricated hydrogel/scaffold systems and avoids the need for potentially damaging ultraviolet irradiation as required for many PEG-based systems that can be crosslinked in situ.

However, PNIPAAM homopolymers suffer from syneresis (e.g., hydrogel deswelling/hydrophobic collapse), lack of biodegradability, and lack of inherent mechanisms for drug loading and/or environmentally-triggered release. More recently, biodegradable variants of PNIPAAM have been reported, though these materials still lack mechanisms for controlled, in situ drug release. Furthermore, ABC triblock polymer-based micelles for formation of thermo-responsive hydrogels have been described, but these hydrogels suffer from a lack of biodegradability and “smart” drug release mechanisms.

Hence, there remains a need for an injectable hydrogel platform that has biodegradability and enables sustained, “smart” drug release.

### SUMMARY

The presently-disclosed subject matter meets some or all of the above-identified needs, as will become evident to those of ordinary skill in the art after a study of information provided in this document.

This Summary describes several embodiments of the presently-disclosed subject matter, and in many cases lists variations and permutations of these embodiments. This Summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently-disclosed subject matter, whether listed in this Summary or not. To avoid excessive repetition, this Summary does not list or suggest all possible combinations of such features.

The presently-disclosed subject matter includes a polymer (i.e., copolymer) comprising a thermally responsive block and a hydrophobic block. In some embodiments the copolymer is a terpolymer. Specific embodiments include a thermo-responsive, ROS degradable ABC triblock terpolymer comprising poly(propylenesulfide)-block-poly(N,N-dimethylacrylamide)-block-poly(N-isopropylacrylamide) (PPS₆₀-b-PDMA₁₅₀-b-PNIPAAM₁₅₀).

Thus, the present polymer overcomes many of the problems associated with basic, PNIPAAM-based thermoresponsive hydrogels, and provides a novel platform for sustained, cell-mediated drug release from an injectable hydrogel and/or sustained therapy to encapsulated cells.

### BRIEF DESCRIPTION OF THE DRAWINGS

FIG. 1 is a schematic view of micelle formation and transition to a hydrogel.

FIG. 2 is a graph showing that a hydrogel reduces the concentration of H₂O₂ in excisional rat wounds.

FIG. 3A is a graph showing MIN6 aggregates with various gels in normal culture.

FIG. 3B is a graph showing MIN6 aggregates with various gels in culture with 100 μM H₂O₂.

FIG. 4 is a schematic view of a method for forming a hydrogel over seeded islet cells.

FIG. 5 is a graph showing the protective effects of a hydrogel on islet cells exposed to reactive oxygen species.

FIG. 6 is a schematic view of a model for texting insulin-producing cell transplant for type 1 diabetes.

FIG. 7 is a schematic view of synthesis of ROS-degradable, temperature-responsive PPS₆₀-b-PDMA₁₅₀-b-PNIPAAM₁₅₀ triblock copolymer via anionic and RAFT polymerization.

FIG. 8 is a graph showing GPC traces of PPS₆₀-OH, PPS₆₀-CEP, PPS₆₀-b-PDMA₁₅₀-CEP, and PPS₆₀-b-PDMA₁₅₀-b-PNIPAAM₁₅₀-CEP.

FIG. 9A is a graph showing DLS-based size measurement of PPS₆₀-b-PDMA₁₅₀-CEP and PPS₆₀-b-PDMA₁₅₀-b-PNIPAAM₁₅₀-CEP micelles at 1 mg/mL concentration in DPBS (pH 7.4).

FIG. 9B is a TEM image of PPS₆₀-b-PDMA₁₅₀-b-PNIPAAM₁₅₀-CEP at 1 mg/mL concentration at 25° C.

FIG. 10A is a graph showing LCST measurement of PDN at 1 wt % concentration in DPBS at pH 7.4 at 500 nm wavelength with a heating rate of 1° C./min.

FIG. 10B is a graph showing measurement of storage (G′) and loss modulus (G″) as a function of temperature for terpolymer solutions at 2.5 wt % with a heating rate of 1° C./min at ω=10 rad/sec frequency and 1% strain. The black arrow indicates the LCST value for the respective polymer concentration.

FIG. 10C is a graph showing measurement of storage (G′) and loss modulus (G″) as a function of temperature for terpolymer solutions at 5.0 wt % with a heating rate of 1° C./min at ω=10 rad/sec frequency and 1% strain. The black arrow indicates the LCST value for the respective polymer concentration.

FIG. 10D is a graph showing measurement of storage (G′) and loss modulus (G″) as a function of temperature for terpolymer solutions at 7.5 wt % with a heating rate of 1° C./min at ω=10 rad/sec frequency and 1% strain. The black arrow indicates the LCST value for the respective polymer concentration.

FIG. 11A is a graph showing measurement of storage (G′) and loss modulus (G″) as a function of temperature for a 5 wt % terpolymer concentration in the presence of SIN-1 (1 mM) with a heating rate of 1° C./min at ω=10 rad/sec frequency and 1% strain.

FIG. 11B shows that the terpolymer is soluble at room temperature, gels after 30 sec at 37° C., and destabilizes after overnight incubation with 0.5 M H₂O₂.

FIG. 12 is a graph showing in vitro H₂O₂-dependent drug release kinetics from Nile red-loaded hydrogels (5 wt % terpolymer concentration) in PBS (pH 7.4) at 37° C. To access ROS-dependent drug release, hydrogel samples were incubated with 1, 100, and 500 mM H₂O₂ over a 64 h time course.

FIG. 13 is a graph showing in vitro cytotoxicity evaluation of NIH 3T3 mouse fibroblasts encapsulated into 5 wt % terpolymer hydrogels.

FIG. 14A is a ¹H NMR spectra of PPS₆₀-OH.

FIG. 14B is a ¹H NMR spectra of macro-CTA PPS₆₀-CEP.

FIG. 14C is a ¹H NMR spectra of diblock copolymer PPS₆₀-b-PDMA₁₅₀-CEP.

FIG. 14D is a ¹H NMR spectra of triblock copolymer PPS₆₀-b-PDMA₁₅₀-b-PNIPAAM₁₅₀-CEP in CDCl₃.

FIG. 14E is a ¹H NMR spectra of triblock copolymer PPS₆₀-b-PDMA₁₅₀-b-PNIPAAM₁₅₀-CEP in D₂O at 25° C. suggesting formation of micelles with PPS core.

FIG. 14F is a ¹H NMR spectra of triblock copolymer PPS₆₀-b-PDMA₁₅₀-b-PNIPAAM₁₅₀-CEP in D₂O at 37° C. showing only peaks corresponding to PDMA protons indicating hydrophobic PPS and PNIPAAM domains in hydrogels.

FIG. 15 shows representative photos of terpolymer solution in DPBS (pH 7.4) at 2, 2.5. 5.0 and 7.5 wt % concentrations at 25 and 37° C. The terpolymer solutions formed stable hydrogels at and above 2.5 wt % concentration at 37° C.

FIG. 16 is a graph showing measurement of storage (G′) and loss modulus (G″) as a function of frequency for 5.0 wt % terpolymer solution at 37° C. with 1% strain.

FIG. 17A shows representative images from IVIS imaging to monitor local drug retention after subcutaneous injection of 50 μL of dye-loaded triblock polymer solution (blue circle, top left) and dye-loaded diblock copolymer solution (green circle, bottom right).

FIG. 17B is a graph showing quantification of in vivo cumulative drug release from the drug-loaded triblock copolymer hydrogels and diblock micelles.

### DESCRIPTION OF EXEMPLARY EMBODIMENTS

The details of one or more embodiments of the presently-disclosed subject matter are set forth in this document. Modifications to embodiments described in this document, and other embodiments, will be evident to those of ordinary skill in the art after a study of the information provided in this document. The information provided in this document, and particularly the specific details of the described exemplary embodiments, is provided primarily for clearness of understanding and no unnecessary limitations are to be understood therefrom. In case of conflict, the specification of this document, including definitions, will control.

While the terms used herein are believed to be well understood by one of ordinary skill in the art, definitions are set forth herein to facilitate explanation of the presently-disclosed subject matter.

Unless defined otherwise, all technical and scientific terms used herein have the same meaning as commonly understood by one of ordinary skill in the art to which the presently-disclosed subject matter belongs. Although any methods, devices, and materials similar or equivalent to those described herein can be used in the practice or testing of the presently-disclosed subject matter, representative methods, devices, and materials are now described.

Following long-standing patent law convention, the terms “a”, “an”, and “the” refer to “one or more” when used in this application, including the claims. Thus, for example, reference to “a cell” includes a plurality of such cells, and so forth.

Unless otherwise indicated, all numbers expressing quantities of ingredients, properties such as reaction conditions, and so forth used in the specification and claims are to be understood as being modified in all instances by the term “about”. Accordingly, unless indicated to the contrary, the numerical parameters set forth in this specification and claims are approximations that can vary depending upon the desired properties sought to be obtained by the presently-disclosed subject matter.

As used herein, the term “about,” when referring to a value or to an amount of mass, weight, time, volume, concentration or percentage is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed method.

As used herein, ranges can be expressed as from “about” one particular value, and/or to “about” another particular value. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

In one embodiment, the presently-disclosed subject matter includes a polymer, such as, but not limited to, a copolymer. In another embodiment, the polymer includes a thermally responsive block and a hydrophobic block. In some embodiments the copolymer is a terpolymer. For example, the terpolymer may include a thermo-responsive, reactive oxygen species (ROS) degradable ABC triblock terpolymer comprising poly(propylenesulfide)-block-poly(N,N-dimethylacrylamide)-block-poly(N-isopropylacrylamide), (“PPS₆₀-b-PDMA₁₅₀-b-PNIPAAM₁₅₀”).

According to one or more of the embodiments disclosed herein, the PPS portion (or “A” block) forms ROS-sensitive hydrophobic nano-domains in aqueous solutions which can be preloaded with hydrophobic drugs and provide sustained, ROS-dependent drug delivery following in situ hydrogel formation. Upon exposure to ROS, the hydrophobic PPS goes through a two-stage transition to more hydrophilic poly(propylene sulphoxide) and ultimately poly(propylene sulphone).^{[10]} This PPS reaction with reactive oxygen species is irreversible and/or may have a cell-protective antioxidant effect on encapsulated cells or other cells and tissues in the vicinity of the hydrogel. In some embodiments, this phase change is utilized to trigger nanoparticle disassembly and “smart” drug release.^{[11]} Hydrophilic DMA is a biocompatible, neutral hydrophilic block that maintains hydration of the polymer and serves as the middle “B” block to ensure formation of non-syneresing, cytocompatible hydrogels.^{[12]} In some embodiments, the DMA is replaced with other neutral, hydrophilic monomers and/or hydrophilic zwitterionic monomers such as, but not limited to, oligoethylene glycol acrylate or methacrylate, hydroxypropyl methacrylamide (HPMA), 2-Methacryloyloxyethyl phosphorylcholine, or any other suitable replacement. PNIPAAM forms the terpolymer's “C” block owing to its solubility in water at room temperature and ability to forms aggregates when heated above its LCST, which induces supramolecular assembly into non-cytotoxic, non-syneresing hydrogels.

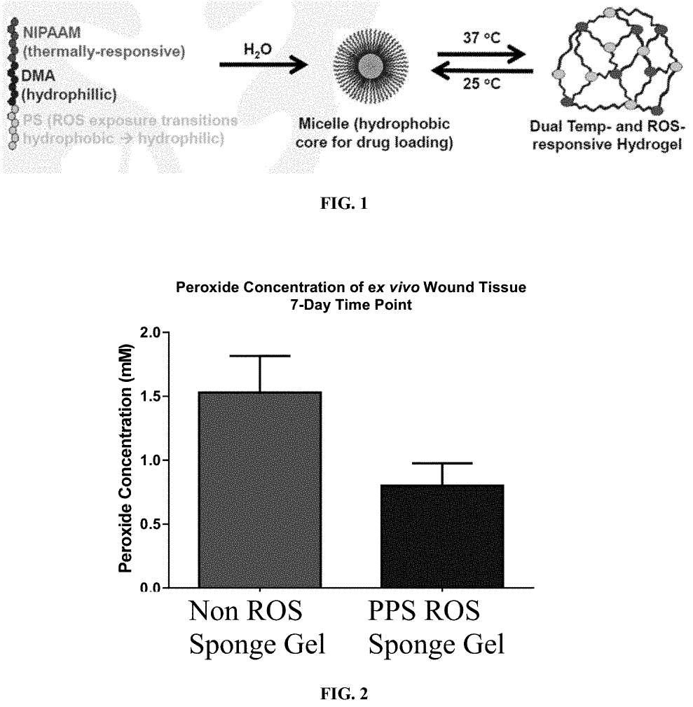

In one embodiment the polymer is injectable at temperatures below physiological temperature, such as, but not limited to, room temperature of about 25° C. In another embodiment, the injectable polymer is synthesized and employed to form physically cross-linked hydrogels in situ. In some embodiments, these injectable, in situ forming physically cross-linked hydrogels provide ROS-triggered active agent release. For example, as illustrated in FIG. 1, specific triblock polymers may assemble into stable micelles in aqueous solution at 25° C. and undergo a transition to a hydrogel when the micelle solution at or above 2.5 wt % concentration is heated to 37° C. (i.e., body temperature). The formation of stable micelles at ambient temperatures and transition to a hydrogel at elevated temperatures decreases or eliminates the practical manufacturing and storage issues of prefabricated hydrogel/scaffold systems. Additionally, the formation of stable micelles at ambient temperatures and transition to a hydrogel at elevated temperatures decreases or eliminates the use of potentially damaging ultraviolet irradiation or addition of cytotoxic reagents required for many PEG based systems that can be cross-linked in situ. Without wishing to be bound by theory or mechanism, this “physical” hydrogel crosslinking is believed to be driven by the lower critical solution temperature (LCST) behavior of PNIPAAM that causes it to switch from water soluble to a more hydrophobic, self-aggregating state above its LCST.

In some embodiments, the micelles include a hydrophobic PPS core capable of being loaded with hydrophobic small molecule drugs (i.e., active agent), and an outer micelle corona formed from the thermoresponsive PNIPAAM polymer block. Accordingly, in some embodiments, the present polymer, which is also referred to herein as a hydrogel, forms an active agent depot that provides ROS-dependent active agent release. The ROS-dependent active agent release includes, but is not limited to, sustained and/or “on demand” release of any suitable hydrophobic small molecule drug, antioxidant, anti-inflammatory drug, or combination thereof. For example, the hydrophobic (e.g., PPS) polymer block, which forms the core of the micelles when they assemble at room temperature, is converted from hydrophobic to a more hydrophilic state upon exposure to ROS. This conversion of the hydrophobic polymer block to a more hydrophilic state provides a mechanism for both ROS-dependent active agent (e.g., drug) release and hydrogel degradation. As compared to existing matrix metalloproteinase (MMP)-cleavable peptides, this hydrogel degradation mechanism is more generalizable. Additionally or alternatively, in some embodiments, this active agent release and/or hydrogel degradation provides sustained, on-demand delivery of at least one active agent.

In one embodiment, the sustained release of the active agent provides extended delivery of the active agent to an encapsulated cell. For example, the sustained release may provide extended drug delivery to encapsulated islet cells in vivo. In another embodiment, the sustained release provides extended drug delivery to the encapsulated cells over a timeframe sufficient for resolving inflammation and/or for donor and host tissues to reach a vascularized and well-integrated steady state. Suitable timeframes for the sustained release of the at least one active agent include, but are not limited to, a period of at least 24 hours, at least 1 weeks, at least 2 weeks, up to 10 weeks, up to 8 weeks, between 24 hours and 10 weeks, between 2 days and 8 weeks, between 3 days and 8 weeks, between 4 days and 8 weeks, between 5 days and 8 weeks, between 6 days and 8 weeks, between 1 and 8 weeks, between 2 and 8 weeks, between 4 and 8 weeks, between 6 and 8 weeks, or any combination, sub-combination, range, or sub-range thereof.

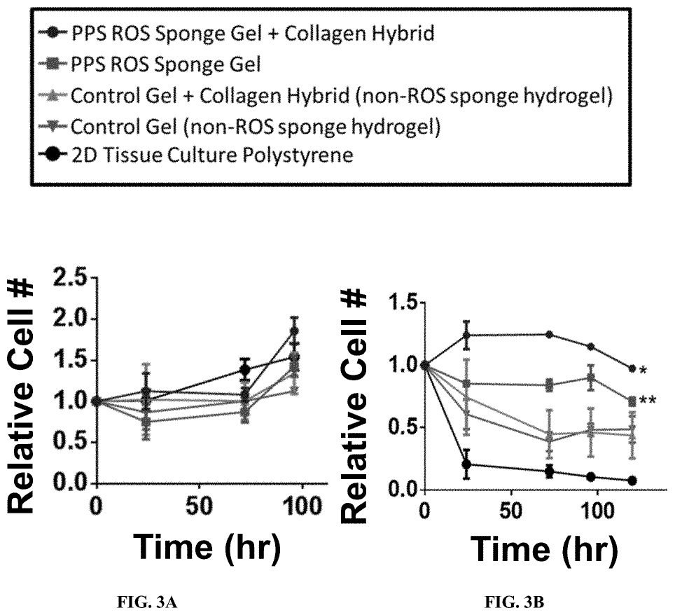

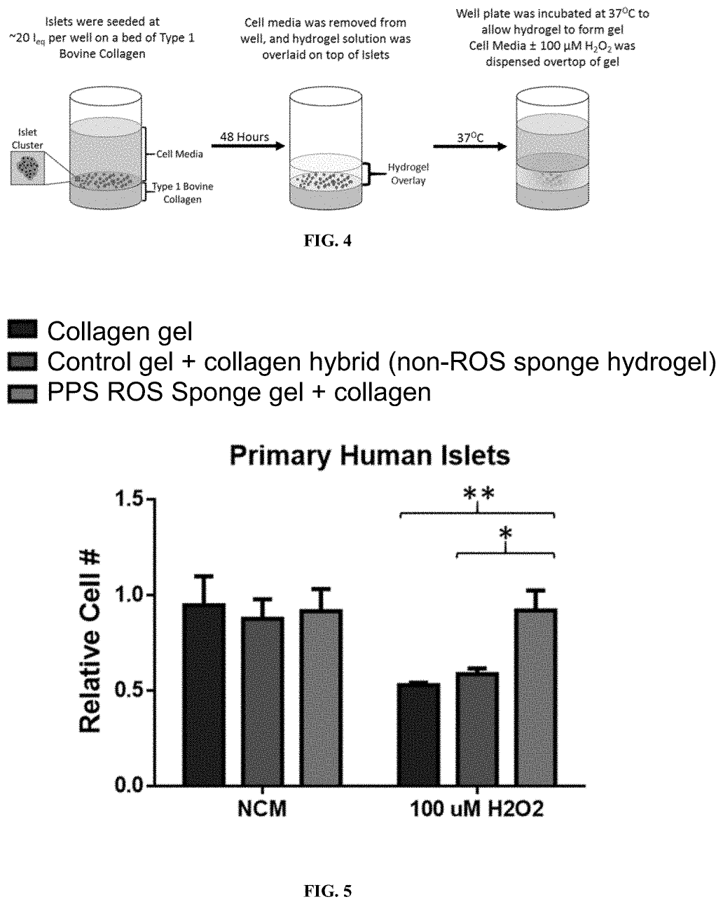

Embodiments of the present hydrogels show minimal in vitro cytotoxicity. In some embodiments, the hydrogel provides a hydrogel-based cell microenvironment that pharmaceutically modifies the host inflammatory response during initial implantation, promotes rapid vascular in-growth, and/or slowly degrades as ECM is formed and donor and host tissues integrate. For example, in one embodiment, as illustrated in FIG. 2, the hydrogel reduces the concentration of ROS in excisional rat wounds. In another example, as illustrated in FIGS. 3-5, the hydrogel protects encapsulated cells from ROS toxicity. As compared to the decreased cell numbers when exposed to ROS in FIG. 3, the islets cells in FIG. 5 show increased cell numbers when encapsulated in the hydrogel according to the method illustrated in FIG. 4. More specifically, after seeding the islet cells on a collagen bed, removing the media, overlaying a hydrogel solution on top of the islets, and incubating the hydrogel solution to form the hydrogel (FIG. 4), the islets exhibited increased viability/cell survival when exposed to ROS (simulated by H₂O₂) (FIG. 5).

Accordingly, in some embodiments, the hydrogels are used for cell encapsulation and sustained in situ drug release to encapsulated cells. For example, embodiments of the present polymer may be used to deliver/encapsulate various types of cells, cell therapies (e.g., stem cells, pancreatic islets), and/or therapeutics to tissue or encapsulated cells under oxidative environment. The encapsulation of cells and sustained release of active agents from the hydrogels may increase cell viability and/or provide increased cell functionality with decreased cell delivery.

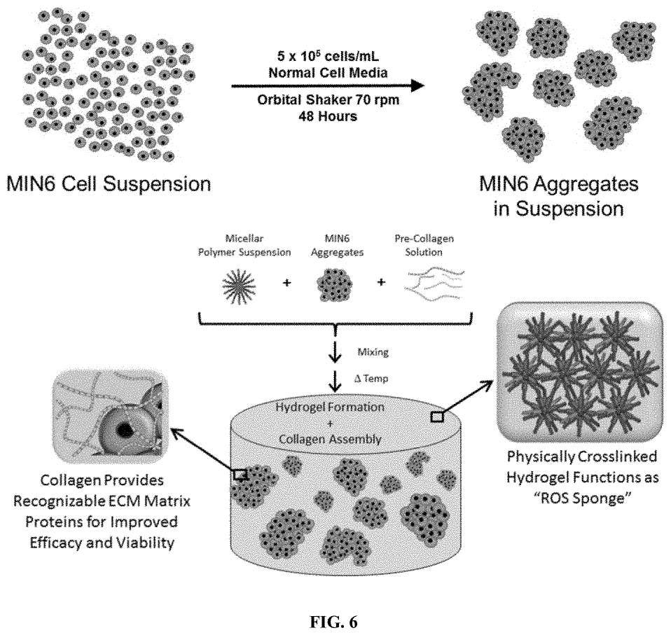

In one embodiment, the hydrogel disclosed herein provides sustained in situ release of the antioxidant curcumin to encapsulated islets post-transplant for the treatment of type 1 diabetes. Without wishing to be bound by theory, the curcumin is believed to provide both antioxidant and anti-inflammatory functions, which protect the encapsulated cells from oxidative stresses. In type 1 diabetes, the sustained release of curcumin is believed to act as a prophylactic, protecting islets against STZ-induced death in vitro and in vivo. For example, in some embodiments, the sustained release of curcumin may increase human islet production of antioxidant enzymes, protect islet viability during cryopreservation, decrease inflammation and fibrosis, and/or enhance function of transplanted islets. Additionally or alternatively, the active agent may include a pro-angiogenic drug, such as, but not limited to, a small molecule PHD2 inhibitor. For example, in one embodiment, the active agent in the hydrogel includes both curcumin and one or more PHD2 inhibitors, such as, but not limited to, JNJ-42041935, DFO, and/or DMOG. The sustained delivery of the curcumin and PHD2 inhibitor decreases ROS-induced islet apoptosis and/or increases graft neovascularization. In another embodiment, these effects act synergistically to improve performance of islets transplanted into type 1 diabetic mammals. Furthermore, as illustrated in FIG. 6, the hydrogels and/or encapsulated cells may be delivered with a cell support material, such as, but not limited to, an extra cellular matrix (ECM), collagen, or any other suitable cell support.

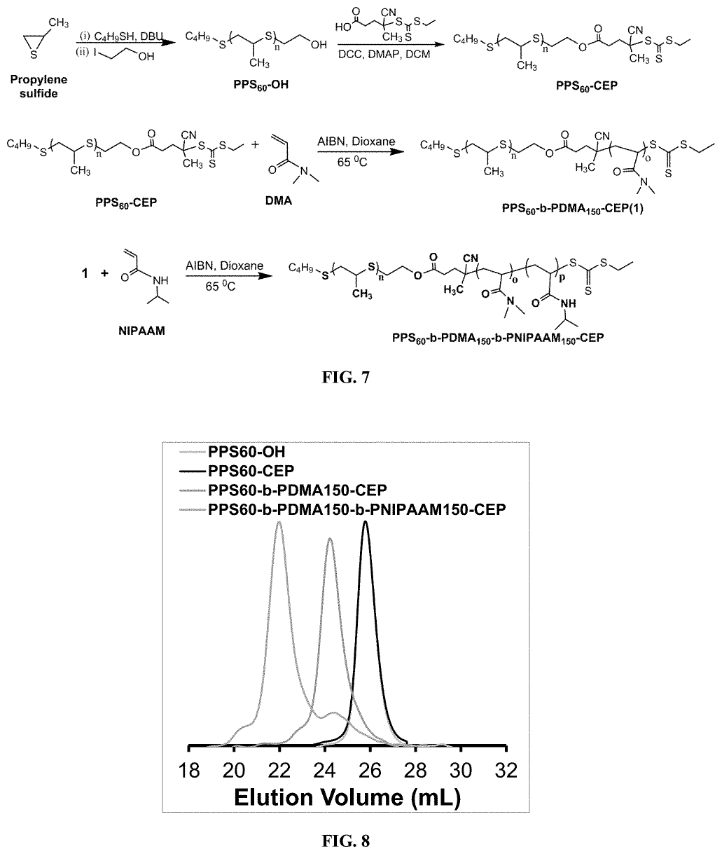

In some embodiments, the synthesis of an ABC triblock polymer includes a combination of anionic and reversible addition-fragmentation chain transfer (RAFT) polymerization. For example, in one embodiment, as illustrated in FIG. 7, the first step in synthesis of the PPS₆₀-b-PDMA₁₅₀-b-PNIPAAM₁₅₀ (PDN) terpolymer includes anionic polymerization of propylene sulfide. Next, the propagation of the PPS chain polymerization is quenched by the addition of 2-iodoethanol to introduce hydroxyl groups at the terminal ends of the PPS. The quenching of the PPS chain polymerization with 2-iodoethanol forms a PPS-based RAFT macro-chain transfer agent (CTA). PPS-OH is then coupled with the RAFT CTA 4-Cyano-4-(ethylsulfanyltiocarbonyl) sulfanylpentanoic acid (ECT) using standard DCC/DMAP coupling. The PPS-ECT is then employed for RAFT polymerization of DMA to form a diblock copolymer, followed by copolymerization of the generated PPS-b-PDMA diblock macro-CTA with NIPAAM to form the triblock polymer.

In some embodiments, the PPS-ECT RAFT macro-chain transfer agent provides a combination of the desirable properties of PPS with the highly-controlled synthesis technique RAFT, which is suitable for use with a diversity of monomer chemistries, including formation of thermo-responsive PNIPAAM. Accordingly, as will be appreciated by those skilled in the art, the ABC triblock polymer is not limited to the example above and may include any other suitable combination of monomers. Additionally, as will also be appreciated by those skilled in the art, the molecular weight of the resulting polymer may be modified by adjusting the synthesis conditions described herein. For example, using the method disclosed above, various PPS-b-PDMA-b-PNIPAAM polymer may be formed, including, but not limited to, PPS₅₀-b-PDMA₁₅₀-b-PNIPAAM₁₅₀, PPS₆₀-b-PDMA₁₅₀-b-PNIPAAM₁₅₀, PPS₅₀-b-PDMA₂₅₀-b-PNIPAAM₁₅₀, or any other suitable molecular weight polymer. In view thereof, in some embodiments, the architecture of the polymer is adjusted to modify hydrogel biomechanics, drug loading, degradation kinetics, drug release kinetics, cytocompatibility, or a combination thereof.

The presently-disclosed subject matter is further illustrated by the following specific but non-limiting examples. The following examples may include compilations of data that are representative of data gathered at various times during the course of development and experimentation related to the present invention.

### EXAMPLES

### Example 1

ROS was chosen as the biological stimuli to promote drug delivery and hydrogel degradation due to their natural production over a wide range of physiological events^{[7]}. The overproduction of ROS is closely related to the development and progression of many pathophysiological diseases, including as atherosclerosis, aging, diabetes, and cancer^{[8]}. As a result, ROS-responsive delivery platforms are desirable for delivery of small molecule drugs to diseased sites by targeting oxidative microenvironments^{[9]}.

FIG. 7 outlines the synthesis steps involved in preparation of the PPS₆₀-b-PDMA₁₅₀-b-PNIPAAM₁₅₀ (PDN) terpolymer. The first step involves anionic polymerization of propylene sulfide using 1-butanethiol as an initiator in the presence of 1,8-Diazabicyclo[5.4.0]undec-7-ene(DBU) in THE at 0° C. for 2 h.

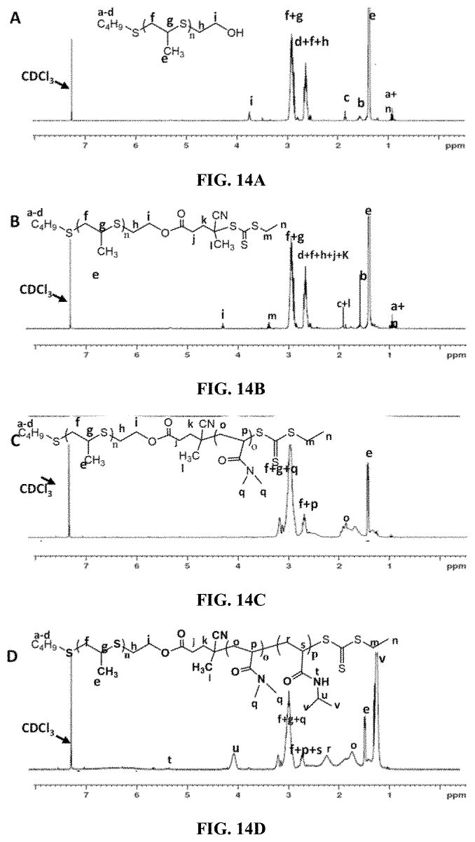

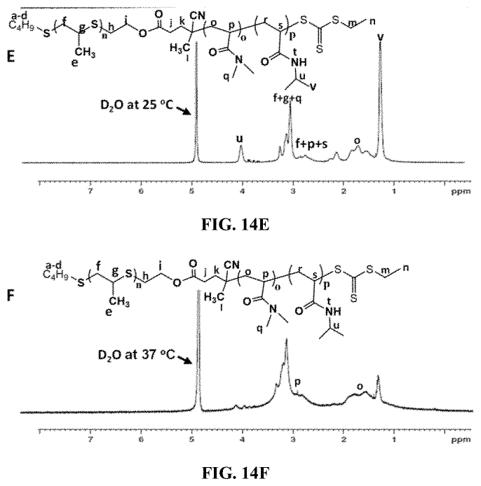

The propagation of the PPS chain polymerization was quenched by the addition of 2-iodoethanol to introduce hydroxyl groups at the terminal ends of the PPS, forming a PPS-based reversible addition-fragmentation chain transfer (RAFT) macro chain transfer agent (CTA). The hydroxyl functionalization of PPS was confirmed by ¹H NMR spectroscopy with the appearance of the CH₂ proton peak at 3.75 ppm (FIG. 14A). PPS₆₀-OH was coupled with the RAFT CTA 4-Cyano-4-(ethylsulfanyltiocarbonyl) sulfanylpentanoic acid (ECT) using standard DCC/DMAP coupling.^{[13]} The conjugation of ECT to PPS₆₀-OH was confirmed by ¹H NMR as the CH₂ proton peak at 3.75 ppm shifted to 4.2 ppm, the characteristic peak designating ester formation (FIG. 14B). The ¹H NMR spectra of PPS₆₀-CEP showed an 81% conjugation of CEP onto PPS₆₀-OH as calculated from the ratio of the CH₂ proton peak at 3.4 ppm to the PPS methyl proton peak at 1.35 ppm.

The synthesized PPS₆₀-ECT was then employed for the RAFT polymerization of DMA using AIBN in dioxane at 65° C., for 24 h. The clear shift in the gel permeation chromatography (GPC) trace and the presence of characteristic PDMA peaks in the ¹H NMR spectra indicated the successful formation of the diblock copolymer (FIG. 14C). The generated PPS₆₀-b-PDMA₁₅₀ diblock macro-CTA was utilized for triblock copolymerization with NIPAAM in dioxane at 65° C. using AIBN for 9 h (FIG. 14D). The GPC traces demonstrated a unimodal polymer size distribution and low PDI values, indicating a high blocking efficiency and a controlled polymerization (FIG. 8). The full polymer characterization data are seen in Table 1.

Previous attempts to use RAFT polymerization for the preparation of PPS based blocked polymers have employed the thioacyl group transfer (TAGT) method in combination with RAFT polymerization by using dithiobenzoate as a CTA^{[14]}. Our attempts to prepare a PPS₆₀-b-PDMA₁₅₀ block copolymer by RAFT polymerization using a PPS macro CTA prepared by the TAGT method resulted in high molecular weight polymers with relative broad polydispersity values. To overcome this limitation in the polymerization scheme, we developed a novel methodology to prepare a PPS-based RAFT macro CTA by DCC/DMAP mediated coupling between PPS₆₀-OH and CEP, which was then used to prepare a well-defined PPS₆₀-b-PDMA₁₅₀-b-PNIPAAM₁₅₀-CEP triblock copolymer with a controlled, well-defined molecular weight (Table 1).

TABLE 1
Molecular weight data of prepared polymers
via anionic and RAFT polymerization.

| S.N. | Polymer | M_{n, Th}^{a} | M_{n, NMR}^{b} | M_{n, GPC}^{c} | PDI^{d} |
|---|---|---|---|---|---|
| 1. | PPS₆₀-OH, | 4,500 | 4,445 | 4,200 | 1.33 |
| 2. | PPS₆₀-CEP | 4,762 | 4,729 | 4,509 | 1.33 |
| 3. | PPS₆₀-b-PDMA₁₅₀-CEP | 19,762 | 20,629 | 19,800 | 1.15 |
| 4. | PPS₆₀-b-PDMA₁₅₀-b- | 36,712 | 37,127 | 37,462 | 1.19 |
| | PNIPAAM₁₅₀-CEP | | | | |

^{a}Theoretical molecular weight calculated based on monomer conversion,
^{b}Molecular weight based on ¹H NMR analysis,
^{c}Number average molecular weight determined by GPC analysis, and
^{d}polydispersity index determined by GPC analysis.

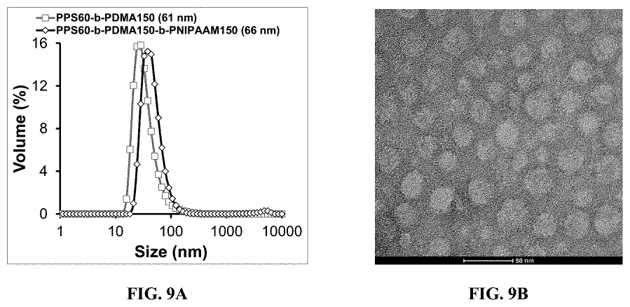

Following characterization of the base polymers, the micelle formation ability of the diblock PPS60-b-PDMA₁₅₀-CEP was tested before the addition of the NIPAAM “C” block to ensure that the PPS core provided sufficient hydrophobicity for driving micelle formation. As predicted, the hydrophobic PPS portion of both the diblock and triblock polymers allowed for self-assembly into stable micelles in an aqueous medium. Micelle formation was confirmed by dynamic light scattering (DLS)-based size measurements and transmission electron microscopy (TEM) as seen in FIG. 9, the size distribution of the respective diblock and triblock copolymer micelles at a 1 mg/mL concentration in DPBS (pH 7.4), while the TEM image of the triblock copolymer micelles at 25° C. demonstrates good agreement with the quantitative DLS size measurements (FIG. 9B). The diblock and triblock copolymers formed stable micelles with an average diameter of 61 and 66 nm, respectively (FIG. 4A). In principle, ABC terpolymers produce stable core-shell structures in aqueous solutions at RT but allow for the formation of more stable and ordered 3D hydrogels once heated above the terpolymer's LCST^{[6c]}. Therefore, ABC terpolymers produce gels with a sharper gel transition at relatively low polymer concentrations when compared to randomly ordered ABA triblock copolymers^{[6a]}.

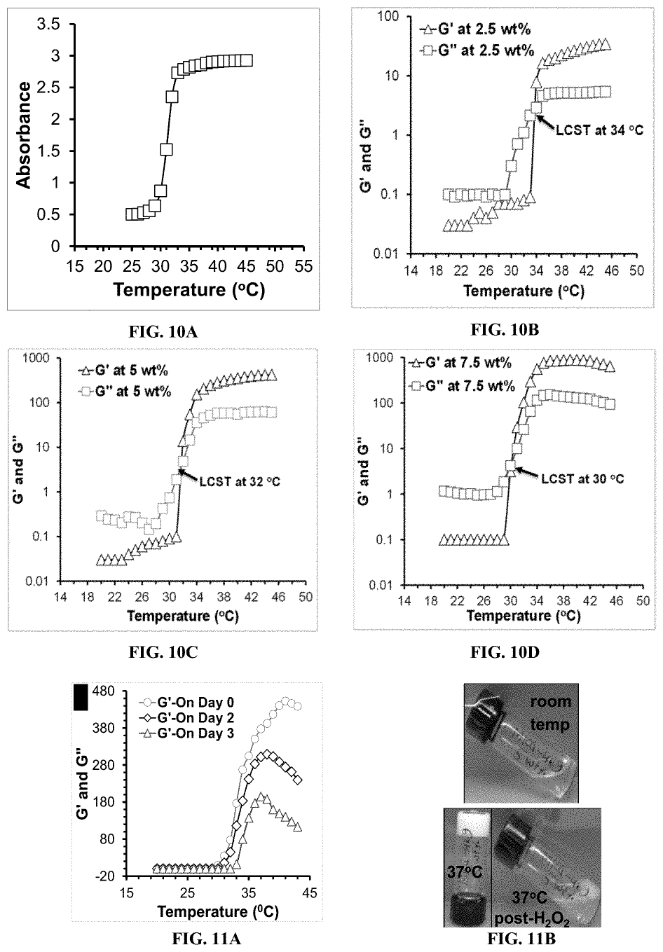

To determine the thermo-responsive behavior of the micelles in an aqueous solution, the LCST of the terpolymer (1 wt % concentration) was determined by measuring the UV-based absorption from 20° C. to 45° C. (FIG. 10A). Between 30-34° C., the clear polymer solution turned cloudy and exhibited a sharp change in absorption around 30° C. which can be attributed to the well-known thermo-responsive nature of PNIPAAM near 30° C.^{[2]}.

However, the LCST of NIPAAM-based random copolymers can be adjusted by copolymerizing NIPAAM with hydrophobic and hydrophilic monomers^{[15]}. In our design, the terpolymer possesses a permanently hydrophobic PPS block which allows the polymer to self-assemble into stable micelles in an aqueous solution while isolating the extended PNIPAAM chains from any active interaction with the PPS and PDMA portions of the polymer chains. Consequently, this ABC terpolymer demonstrated an LCST value similar to a pure PNIPAAM homopolymer which allows for thermo-reversible gelation in heating/cooling cycles through PNIPAAM's LCST at 30° C.^{[15b]}.

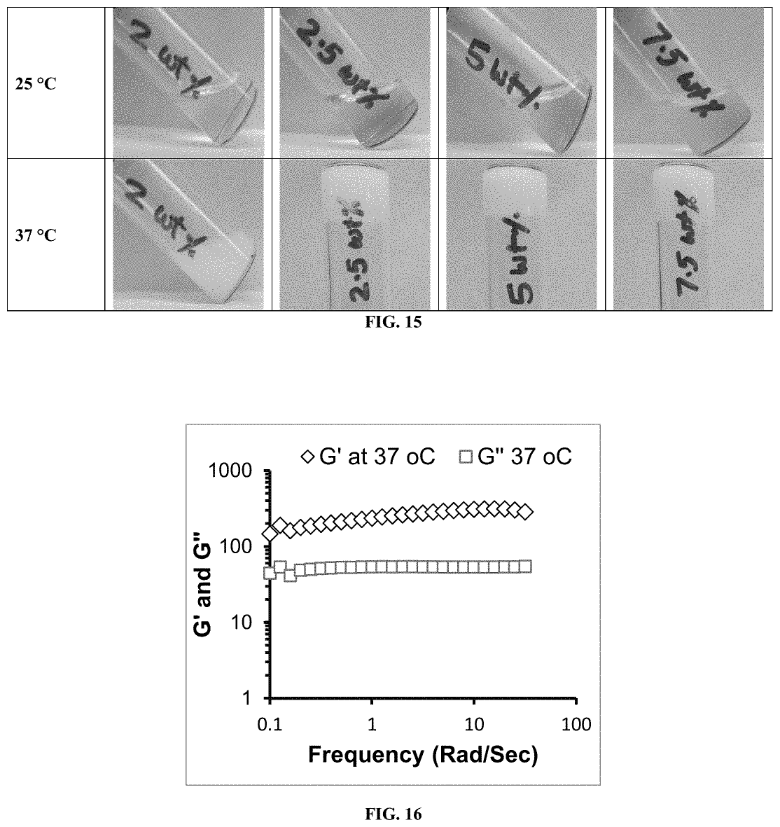

The vial inversion method was used to test the gelation ability of aqueous terpolymer solutions ranging from 2.0 to 7.5 wt % concentrations at 37° C. (FIG. 15). The copolymer solutions transitioned into stable hydrogels within 30 seconds at and above the 2.5 wt % concentration and returned to transparent solutions when cooled to an ambient temperature. The terpolymer solutions at higher concentrations underwent relatively faster gelation transitions, though the lower concentration hydrogels still formed mechanically robust hydrogels.

To further investigate the gelation temperature of the terpolymer solutions and the effect of polymer concentration on the hydrogels' storage and loss moduli (G′ and G″), rheometric temperature sweep measurements were performed on terpolymer solutions over a range from 20 to 45° C. FIG. 10B-D shows the temperature sweep measurement of terpolymer solutions at three different concentrations (2.5, 5.0 and 7.5 wt %) with a heating rate of 1° C./min at a frequency of ω=10 rad/sec and 1% strain. The terpolymer solutions exhibited a sharp increase in both G′ and G″ once heated near their LCST values before the two moduli values crossed over and equilibrated below 37° C., indicating stable hydrogel formation and highlighting the potential utility of this system as an injectable therapeutic material that can solidify once reaching body temperature. The cross over point between G′ and G″ was considered the gelation point. The equilibrium storage moduli (G′) of hydrogels at 2.5, 5.0 and 7.5 wt % concentration were measured at 19, 281 and 851 Pa, respectively (FIG. 10B-D). These data suggest that hydrogel modulus can be tuned by varying terpolymer concentration for tailoring these materials for particular applications. Further highlighting these materials' tunability, terpolymer solutions at 2.5, 5.0 and 7.5 wt % concentration displayed LCST values of 34, 32 and 30° C., respectively. The solutions' LCST was found to decrease with increasing polymer concentration, consistent with previously reported literature^{[16]}. Moreover, a 10 wt % terpolymer solution formed a viscous gel instead of a clear solution at RT, additionally supporting the displayed decrease in LCST with an increase in polymer concentration. Though many PNIPAAM-based hydrogels undergo syneresis and deswelling, the G′ and G″ values of these terpolymer hydrogels did not decrease after reaching 37° C., indicating a lack of syneresis^{[4a]}. The LCST values obtained by rheometry measurements also displayed a close agreement with the UV-based LCST measurement (FIG. 10A).

To verify the linearity of the terpolymer hydrogels' mechanical properties below and above the LCST, oscillatory rheometric measurements^{[6a]} on 5 wt % hydrogels in PBS were carried out over a 0.1-50 rad/sec frequency range (FIG. 16). At these frequencies, the hydrogels demonstrated a consistently higher G″ value at 25° C. while displaying a higher G′ value at 37° C., suggesting the terpolymer solutions behave like a liquid and solid below and above their LCST, respectively.

To investigate ROS-dependent degradation of hydrogels, 3-Morpholinosyndnomine (SIN-1) was used as a model ROS molecule as it has been shown to generate both nitric oxide and superoxide upon decomposition in aqueous solutions^{[17]}. Degradation of 5 wt % hydrogels incubated in SIN-1 (1 mM) over 3 days was confirmed by temperature-dependent rheometry, with G′ values on days 0, 2, and 3 being measured at 378, 301 and 195 Pa at 37° C., respectively (FIG. 11A). In the presence of ROS-generating SIN-1, the hydrogels demonstrated a decrease in modulus over time, which can be attributed to an ROS-mediated oxidative transformation of the hydrophobic PPS into the more hydrophilic poly(sulfone) and ultimately water soluble poly(propylene sulfoxide)^{[11a]}. A vial inversion method was used to access daily hydrogel stability just prior to rheology measurement at 37° C. (FIG. 11B), with hydrogels being stable out to 2 days but falling apart on day 3.

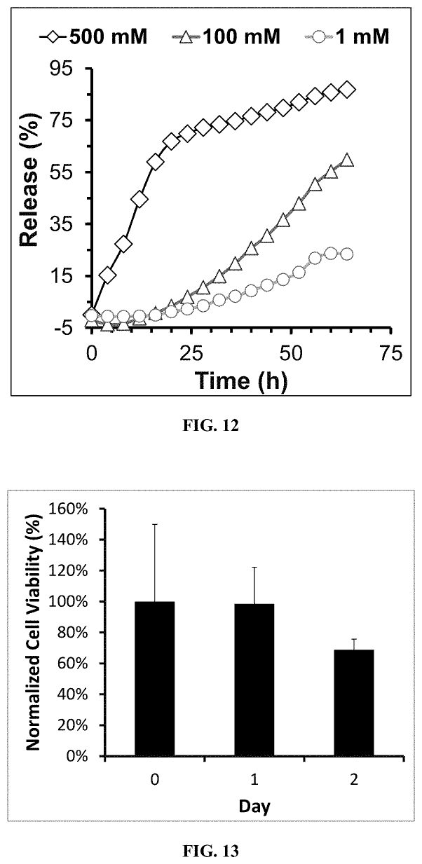

To asses in vitro drug release from the hydrogels, Nile red (used as a model hydrophobic drug^{[18]}) was encapsulated in terpolymer micelles prior to hydrogel formation before incubating the formed gels with H₂O₂ to mimic the presence of a pathophysiologic oxidative microenvironment^{[19]}. FIG. 12 shows the in vitro, H₂O₂-dependent drug release kinetics of Nile red-loaded hydrogels (5 wt %) incubated with H₂O₂ in PBS (1, 100 and 500 mM concentrations) at 37° C. over a 64 h time course. The fluorescence intensity of Nile red-loaded hydrogels treated with H₂O₂ was found to decrease over time, and the rate of this decrease was dependent on the concentration of H₂O₂ present. As with the ROS-mediated hydrogel degradation and mechanical property reduction, the decrease in Nile red fluorescence intensity under oxidative conditions can be explained by the oxidative phase transition of the terpolymer's PPS block from a hydrophobic sulfide to a more hydrophilic sulfone^{[11a]}. These collective results suggest that the terpolymer micelles' PPS core can be successfully drug-loaded to achieve sustained, ROS-mediated drug release from in situ-forming hydrogels.

To assess the cytotoxicity of the terpolymer hydrogels, NIH 3T3 mouse fibroblasts stably transduced to express luciferase were encapsulated into 5 wt % hydrogels and relative cell number was measured based on luciferase activity over 2 days of culture (FIG. 13). Cell-generated bioluminescent signal was not significantly different over the culture period, indicating that the hydrogels are not cytotoxic.

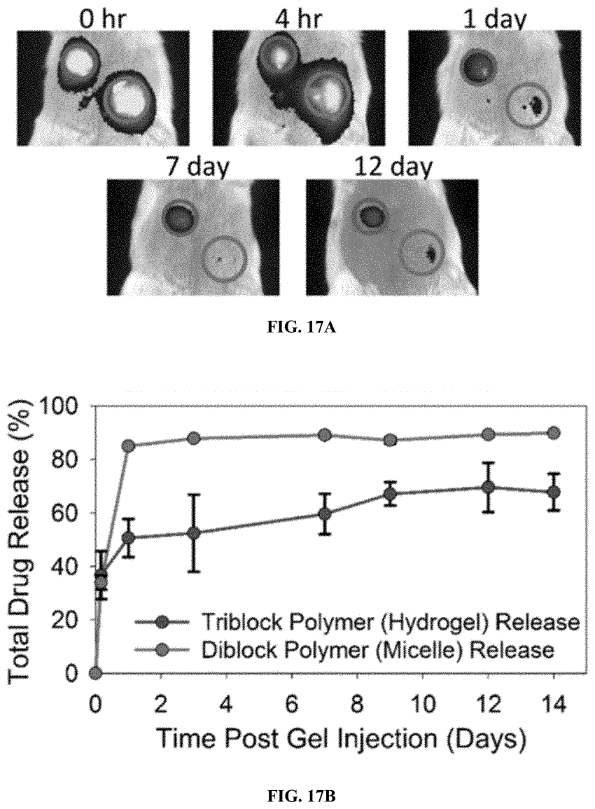

In vivo drug release from subcutaneously injected hydrogels was assessed using the model hydrophobic drug Nile red. The triblock copolymer PPS₆₀-b-PDMA₁₅₀b-PNIPAAM₁₅₀ and the diblock copolymer PPS₆₀-b-PDMA₁₅₀ were preloaded with Nile red, which was imaged over time to determine local retention of the drug based on the controlled release hydrogels. As illustrated in FIGS. 17A and B, the triblock polymer solutions formed robust hydrogels upon subcutaneous injection, with local retention and release of the drug over 14 days. In contrast, the diblock polymer solutions, which did not include the PNIPAAM block, were quickly dispersed from the injection site over 24 hours.

In summary, a novel, ROS-degradable, thermo-responsive ABC triblock terpolymer with a well-controlled molecular weight was synthesized by a combination of anionic and RAFT polymerization. In an aqueous solution at room temperature, the terpolymer dissolved into a clear solution and assembled into stable micelles before ungoing a sharp, reversible thermo-gelation once heated above the polymer solution's LCST value to form stable hydrogels at relatively low concentrations. The terpolymer solutions' LCST were less affected by the presence of hydrophobic/hydrophilic segments, but were dependent on the terpolymer concentration in solution. Temperature-dependent rheometric hydrogel characterization exhibited the materials' sharp, highly temperature-sensitive gelation at relatively low terpolymer concentrations while also displaying the hydrogels' lack of syneresis. ROS-dependent degradation of the hydrogels was demonstrated with by a decrease in the materials' G′ values in the presence of ROS-producing SIN-1, along with the failure of hydrogels to reassemble after overnight incubation with H₂O₂. The terpolymer hydrogels also demonstrated a controlled, sustained, and ROS concentration-dependent release of the model drug Nile red. Finally, the hydrogels exhibited minimal in vitro cytotoxicity with encapsulated fibroblasts. Therefore, these collective data demonstrate the potential utility of these thermo-responsive terpolymers as an injectable platform for cell and drug delivery applications.

### Example 2

With regard to material and methods, briefly, the ABC terpolymer PPS₆₀-b-PDMA₁₅₀-b-PNIPAAM₁₅₀-CEP was synthesized and characterized by GPC (FIG. 8, Table 1) and ¹H NMR spectroscopy (FIG. 14). The formation of micelles at RT was characterized by DLS and TEM (FIG. 9). The LCST values of polymer solutions were measured by UV-based absorption and rheometry (FIG. 10). Hydrogel formation and ROS degradability were tested with the vial inversion method and temperature-dependent rheometry (FIG. 15 and FIG. 10, 11). The release of Nile red from hydrogels was assessed by measuring the Nile red-loaded gels' fluorescence intensity over a 64 h time course (FIG. 12). Hydrogel in vitro cytotoxicity was determined by measuring luciferase activity of encapsulated cells to determine relative cell number over two days in culture (FIG. 13).

More specifically, with regard to the materials, all chemicals were purchased from Sigma-Aldrich (Milwaukee, Wis., USA) unless otherwise noted. Propylene sulfide (PS)(>96%) was purchased from Acros Organics through Fischer Scientific (Pittsburgh, Pa., USA). SIN-1 was purchased from Life Technologies (Grand Island, N.Y., USA) in packages of 1 mg plastic vials. N-Isopropylacrylamide (NIPAAM) was recrystallized twice with n-hexane. 2, 2′-Azoisobutyronitrile (AIBN) was recrystallized from ethanol twice. Propylene sulfide (PS) and N,N-dimethylacrylamide (DMA) was purified by distillation just before polymerization^{[11a]}. 4-Cyano-4-(ethylsulfanyltiocarbonyl) sulfanylpentanoic acid (CEP) was synthesized according to a previously reported procedure^{[20]}.

Synthesis of Hydroxyl End-Functional Poly(Propylene Sulfide) (PPS₆₀-OH)

Poly(propylene sulfide) with a terminal hydroxyl end group was prepared by anionic polymerization of propylenesulfide using DBU/1-buthane thiol as an initiator before subsequent end-capping with 2-iodoethanol. Briefly, in a dried and nitrogen flushed 50 mL RB flask, 1,8-Diazabicyclo[5.4.0]undec-7-ene (DBU) (3 mmol, 0.448 mL) in dry THF (15 mL) was degasses for 30 minutes and reaction mixture temperature was lowered to 0° C. To this flask, a 30 minute degassed solution of 1-butane thiol (1.0 mmol, 0.070 mL) in THF (10 mL) was added drop wise and allowed to react for 30 minutes. Later, freshly distilled and degassed propylene sulfide (60 mmol, 4.68 mL) monomer was added to the reaction mixture and the temperature was maintained at 0° C. for 2 h. The reaction was quenched by addition of 2-Iodoethanol (2 mmol, 0.40 g) and stirred overnight at RT. The next day, the polymer solution was filtered to remove precipitated salt and further purified by three precipitations into cold methanol before vacuum-drying to yield a colorless viscous polymer. ¹H NMR (400 MHz; CDCl₃, δ): 1.3-1.4 (s, CH₃), 2.5-2.8 (s, —CH), 2.8-3.1 (s, CH₂), 3.72 (t, CH₂—OH). (PPS₆₀-OH, Mn=4,200 g/mol, PDI=1.33).

Synthesis of PPS-Based RAFT Macro CTA (PPS₆₀-CEP)

N,N′-Dicyclohexylcarbodiimide(DCC) (0.49 g, 2.4 mmol) was added to a solution of CEP (0.424 g, 2 mmol) and PPS₆₀-OH (3.36 g, 0.8 mmol), and 4-Dimethylaminopyridine (DMAP) (0.029 g, 0.24 mmol) in anhydrous DCM (20 mL) at 0° C. The reaction mixture was stirred for 24 h at RT. The solution was filtered to remove precipitated dicyclohexyl urea and concentrated under vacuum. The crude reaction mixture was first purified by dialysis against DCM for 24 h to remove free CEP and further purified through double precipitation into cold ethanol. ¹H NMR (400 MHz; CDCl₃, δ): 1.35 (t, 3H, —S—CH₂—CH₃), 1.3-1.4 (s, 3H, CH₃), 1.85 (s-C(CN)—CH₃), 2.4-2.67 (m, —CH₂—CH₂—S), 2.5-2.8 (broad s, S—CH), 2.8-3.1 (broad s, 2H, CH₂), 3.42 (q, —S—CH₂—CH₃), 4.2 (t, —OCH₂—CH₂). (PPS₆₀-CEP, M_{n,GPC}=4,509 g/mol, PDI=1.33)

Synthesis of PPS₆₀-b-PDMA₁₅₀-Macro CTA

The diblock copolymer PPS₆₀-b-PDMA₁₅₀-CEP was synthesized by RAFT polymerization of DMA using AIBN as the initiator with a 20:1^{[7a]}:^{[7b]} molar ratio of macro CTA to AIBN. The PPS₆₀-CEP (0.585 g, 0.13 mmol, M_{n,GPC}=4,509), DMA (1.28 mL, 13 mmol), AIBN (1.09 mg, 0.0065 mmol), and dioxane (5 mL) were placed in a dry ampoule, and the solution was degassed by bubbling of ultrahigh purity nitrogen for 30 min. The polymerization was performed at 65° C. for 16 h. The final polymerization mixture was precipitated twice into cold diethyl ether and dried under vacuum at RT to yield a yellow-colored polymer. ¹H NMR (400 MHz; CDCl₃, δ): 1.3-1.4 (s, CH₃ in PPS block), 1.2-1.75 (—CH₂ backbone PDMA), 2.5-2.7 (—CH backbone PDMA), 2.5-2.8 (broad s, CH in PPS block), 2.8-3.1 (broad s, CH₂ next to S), 2.9-3.3 (dimethyl group PDMA), 3.72 (s, CH₂ in ester) ppm. (PPS₆₀-b-PDMA₁₅₀-CEP, M_{n,GPC}=19,800 g/mol, PDI=1.15)

Synthesis of PPS₆₀-b-PDMA₁₅₀-b-PNIPAAM₁₅₀-CEP

PPS₆₀-b-PDMA₁₅₀-CEP was used as macro CTA to build the third block of poly(NIPAAM) with degree of polymerization of 150. PPS₆₀-b-PDMA₁₅₀-CEP (1.27 g, 0.067 mmol), NIPAAM (1.70 g, 15.07 mmol), AIBN (1.12 mg, 0.0067 mmol), and dioxane (5 mL) were placed in a dry glass ampoule equipped with three way stopcock, and the solution was degassed by bubbling with ultrahigh purity nitrogen for 30 min. The ampoule was submerged into a preheated oil bath at 65° C. for 9 h. The polymerization was quenched by exposing the polymer solution to air and the resultant triblock copolymer was precipitated twice into excess cold diethylether. ¹H NMR (400 MHz; CDCl₃, δ): 1.1 (CH₃, PNIPAAM), 1.3-1.4 (s, CH₃ in PPS block), 1.2-1.75 (—CH₂ backbone PDMA), 1.5 (CH₂, PNIPAAM backbone), 1.9 (CH in main chain, PNIPAAM), 2.5-2.7 (—CH backbone PDMA), 2.5-2.8 (broad s, CH in PPS block), 2.8-3.1 (broad s, CH₂ next to S), 2.9-3.3 (dimethyl group of NMe₂, PDMA), 3.72 (s, CH₂ in ester) 3.8 (CH in side chain, PNIPAAM), and 7.5 8.0 ppm (NH, PNIPAAM) ppm. (PPS₆₀-b-PDMA₁₅₀-b-PNIPAAM₁₅₀-CEP, M_{n,GPC}=37,462 g/mol, PDI=1.19).

Polymer Characterization

¹H NMR spectra of organic compounds and polymers were recorded in CDCl₃ with a Bruker 400 MHz spectrometer. The Molecular weight (Mn) and polydispersity (PDI) of polymers were assessed by gel permeation chromatography (GPC, Agilent Technologies, Santa Clara, Calif., USA) using dimethylformamide (DMF)+0.1 M LiBr mobile phase at 60° C. through three serial Tosoh Biosciences TSKGel Alpha columns (Tokyo, Japan). An Agilent refractive index (RI) and Wyatt miniDAWN TREOS light scattering (LS) detector (Wyatt Technology Corp., Santa Barabara, Calif., USA) were used to calculate absolute molecular weight based on dn/dc values experimentally determined through offline injections into the RI detector.

Preparation and Characterization of Polymer Micelles

To assess the abilities of the diblock copolymer PPS₆₀-b-PDMA₁₅₀-CEP and triblock terpolymer PPS₆₀-b-PDMA₁₅₀-b-PNIPAAM₁₅₀-CEP to form stable micelles, 5 mg of each copolymer were dissolved into 100 uL THF and assembled into micelles through drop-wise addition of 5 mL of DPBS (pH 7.4) through a syringe pump under constant stirring. The micelle solutions (1 mg/mL) were filtered through a 0.20 m syringe filter and used for hydrodynamic diameter (Dₕ) measurements using a Malvern Zetasizer Nano-ZS (Malvern Instruments Ltd, Worcestershire, U.K) equipped with a 4 mW He—Ne laser operating at λ=632.8 nm. TEM samples were prepared by addition of 20 μL of terpolymer solution (1 mg/mL) on TEM grids (Electron Microscopy Sciences, Hatfield, Pa., USA) and blotted dry after 60 seconds to counterstain with 3% urenyl acetate stain (10 μL) for 10 seconds. The grids were dried overnight under vacuum prior to imaging on an FEI Tecnai Osiris TEM operating at 200 kV.

Preparation of Hydrogels

The lyophilized triblock copolymer of PPS₆₀-b-PDMA₁₅₀-b-PNIPAAM₁₅₀-CEP was dissolved into DBPS (pH 7.4) at four different concentrations (2.0, 2.5, 5.0 and 7.5 wt %) to test the ability of the terpolymer to form hydrogels at different concentrations. A vial inversion method was used to demonstrate the hydrogel formation at 37° C. To see ROS-triggered destabilization of hydrogels, PPS₆₀-b-PDMA₁₅₀-b-PNIPAAM₁₅₀-CEP at 5 wt % copolymer concentration was incubated with 0.5 M of hydrogen peroxide overnight at 37° C.

Measurement of LCST by UV/Vis Spectroscopy

The optical absorbance of the triblock terpolymer (1 wt % in DPBS at pH 7.4) was measured at 500 nm wavelength on a Varian Cary 5000 UV-VIS-NIR spectrophotometer equipped with a temperature controller set at a heating rate of 1° C./min measuring between 25-45° C. The terpolymer solutions' LCST value was defined as the temperature when the absorbance reached 50% of the maximum.

Rheometry of Polymer Hydrogels

The measurements of viscoelastic properties of aqueous solutions of the triblock terpolymer were conducted on an AR-G2 rheometer (TA Instruments, New Castle, Del.) under oscillatory shear using standard steel parallel-plate geometry (40 mm diameter plate). Predetermined amounts of terpolymer were dissolved in DPBS (2 mL, pH 7.4) to reach the desired weight % concentration before placing the solutions between 40 cm steel parallel-plate geometry. Rheological properties of terpolymer were examined by oscillatory temperature sweep and frequency sweep measurements. Temperature dependent shear storage (G′) and loss moduli (G″) of the terpolymer solutions at three different concentrations were measured from 20° C. to 45° C. with a heating rate of 1° C./min. The terpolymer moduli were measured at a frequency of 10 rad/s and at a controlled strain of 1%. The intersection point of G″ and G′ was considered the sol-gel transition point of the respective terpolymer solution. Frequency sweep dependent shear storage (G′) and loss moduli (G″) of terpolymer solutions (5 wt %) were measured below (at 25° C.) and above the respective solutions' LCST (at 37° C.) at frequencies in the range of between 0.1-50 rad/s.

Rheometry of Hydrogels in Presence of SIN-1

PPS₆₀-b-PDMA₁₅₀-b-PNIPAAM₁₅₀-CEP terpolymer solution at 5 wt % concentration was incubated with 1 mM SIN-1 to access the ROS-mediated degradation of hydrogels on day 0, 2 and 3. To maintain a constant SIN-1 concentration, hydrogels were incubated daily with a fresh 1 mM dose of SIN-1. The hydrogel stability in the presence of SIN-1 was accessed daily by the vial inversion method. Temperature sweep measurements were performed at a frequency ω=10 rad/s and heating rate of 1° C./min.

In Vitro Nile Red Release from Hydrogels

The lyophilized terpolymer, PPS₆₀-b-PDMA₁₅₀-b-PNIPAAM₁₅₀-CEP (100 mg to create final 5 wt % concentration), and Nile red (5 mg) were dissolved in dichloromethane and left overnight in the dark to evaporate solvent. 2 mL of PBS was added over the polymer-Nile red thin film to allow the polymer to dissolve into solution. After 7 days, the Nile red-loaded hydrogel solution was centrifuged to remove any unloaded Nile red. The Nile red-loaded terpolymer solution was carefully decanted and used for the release study. A total of 50 uL solution was added to each well and incubated at 37° C. in a micro plate reader (Tecan Infinite F500, Männedorf, Switzerland) for 1 h. Pre-warmed PBS at 37° C. (50 uL) with different concentrations of H₂O₂ (500, 100, 1, and 0 mM) was added to the respective wells. The decrease in fluorescence intensity over 64 h was monitored at 37° C. with the micro plate reader with an excitation wavelength of 485 nm and an emission wavelength of 535 nm. The percent drug release from each group was estimated relative to control (H₂O₂ untreated groups).

Cell Viability of Hydrogels

Cytotoxicity of hydrogels was determined by measuring relative cell number over time based on luciferase activity. Lyophilized terpolymer was dissolved in DPBS for 24 h at 5 wt % before use in cytotoxicity experiments. NIH 3T3 mouse fibroblasts stably transfected with a firefly luciferase reporter gene were cultured in Dulbecco's Modified Eagle Medium (DMEM) supplemented with 10% fetal bovine serum (FBS) and 1% penicillin/streptomycin. Cells were passaged and added to the polymer solution at a density of 2.0×10⁵ cells/mL of terpolymer solution. Terpolymer/cell solutions were dispensed into three separate black-walled 96-well culture plates in 50 uL aliquots (n=3 suspensions per plate). Each 50 uL polymer suspension contained 1.0×10∝cells. The plates were placed in a cell culture incubator for 30 min to fully solidify the terpolymer/cell suspensions, after which the plates were removed and fresh culture medium was added on top of the solidified hydrogels. For the day 0 plate, a luciferin substrate was added to the hydrogels' media and after 10 min the cell-containing hydrogels were imaged with an IVIS 200 (Xenogen, Alameda, Calif.) bioluminescence imaging system with an exposure time of 2 min to quantify the luciferase-based bioluminescence signal from each hydrogel's viable cell population. The second two culture plates, respectively used to measure cytotoxicity at 24 h and 48 h, were evaluated using the same protocol as described for the day 0 plate.

Throughout this document, various references are mentioned. All such references are incorporated herein by reference, including the references set forth in the following list:

### REFERENCES

[1] aG. D. Nicodemus, S. J. Bryant, Tissue Eng Part B Rev 2008, 14, 149-165; bY. Li, J. Rodrigues, H. Tomas, Chemical Society Reviews 2012, 41, 2193-2221; cY. Qiu, K. Park, Advanced Drug Delivery Reviews 2001, 53, 321-339; dL. Yu, J. Ding, Chemical Society Reviews 2008, 37, 1473-1481; eA. S. Hoffman, Advanced Drug Delivery Reviews 2002, 54, 3-12.
[2] aS. Fujishige, K. Kubota, I. Ando, The Journal of Physical Chemistry 1989, 93, 3311-3313; bF. M. Winnik, Macromolecules 1990, 23, 233-242.
[3] M. A. Ward, T. K. Georgiou, Polymers 2011, 3, 1215-1242.
[4] aT. Gan, Y. Guan, Y. Zhang, Journal of Materials Chemistry 2010, 20, 5937-5944; bL. Klouda, A. G. Mikos, European Journal of Pharmaceutics and Biopharmaceutics 2008, 68, 34-45.
[5] aZ. Cui, B. H. Lee, C. Pauken, B. L. Vernon, Journal of Biomedical Materials Research Part A 2011, 98A, 159-166; bD.-Q. Wu, F. Qiu, T. Wang, X.-J. Jiang, X.-Z. Zhang, R.-X. Zhuo, ACS Applied Materials & Interfaces 2008, 1, 319-327; cX. Huang, B. R. Nayak, T. L. Lowe, Journal of Polymer Science Part A: Polymer Chemistry 2004, 42, 5054-5066; dZ. Cui, B. H. Lee, B. L. Vernon, Biomacromolecules 2007, 8, 1280-1286; eS. T. Wall, C.-C. Yeh, R. Y. K. Tu, M. J. Mann, K. E. Healy, Journal of Biomedical Materials Research Part A 2010, 95A, 1055-1066; fA. K. Ekenseair, K. W. M. Boere, S. N. Tzouanas, T. N. Vo, F. K. Kasper, A. G. Mikos, Biomacromolecules 2012, 13, 1908-1915; gZ. Ma, D. M. Nelson, Y. Hong, W. R. Wagner, Biomacromolecules 2010, 11, 1873-1881; hS. Cerritelli, C. P. ONeil, D. Velluto, A. Fontana, M. Adrian, J. Dubochet, J. A. Hubbell, Langmuir 2009, 25, 11328-11335; iT. N. Vo, A. K. Ekenseair, F. K. Kasper, A. G. Mikos, Biomacromolecules 2013, 15, 132-142.
[6] aC. Zhou, M. A. Hillmyer, T. P. Lodge, Journal of the American Chemical Society 2012, 134, 10365-10368; bI. Koonar, C. Zhou, M. A. Hillmyer, T. P. Lodge, R. A. Siegel, Langmuir 2012, 28, 17785-17794; cC. Li, N. J. Buurma, I. Haq, C. Turner, S. P. Armes, V. Castelletto, I. W. Hamley, A. L. Lewis, Langmuir 2005, 21, 11026-11033; dS. Reinicke, J. Schmelz, A. Lapp, M. Karg, T. Hellweg, H. Schmalz, Soft Matter 2009, 5, 2648-2657.
[7] aW. F. Liu, M. Ma, K. M. Bratlie, T. T. Dang, R. Langer, D. G. Anderson, Biomaterials 2011, 32, 1796-1801; bJ. R. Martin, M. K. Gupta, J. M. Page, F. Yu, J. M. Davidson, S. A. Guelcher, C. L. Duvall, Biomaterials 2014, 35, 3766-3776; cC. de Gracia Lux, S. Joshi-Barr, T. Nguyen, E. Mahmoud, E. Schopf, N. Fomina, A. Almutairi, Journal of the American Chemical Society 2012, 134, 15758-15764.
[8] C. C. Winterbourn, Nat Chem Biol 2008, 4, 278-286.
[9] S. H. Lee, M. K. Gupta, J. B. Bang, H. Bae, H.-J. Sung, Advanced Healthcare Materials 2013, 2, 908-915.
[10] A. Napoli, M. Valentini, N. Tirelli, M. Muller, J. A. Hubbell, Nat Mater 2004, 3, 183-189.
[11] aM. K. Gupta, T. A. Meyer, C. E. Nelson, C. L. Duvall, Journal of Controlled Release 2012, 162, 591-598; bD. Velluto, D. Demurtas, J. A. Hubbell, Molecular Pharmaceutics 2008, 5, 632-642.
[12] Z. Ge, Y. Zhou, Z. Tong, S. Liu, Langmuir 2011, 27, 1143-1151.
[13] C. E. Nelson, J. R. Kintzing, A. Hanna, J. M. Shannon, M. K. Gupta, C. L. Duvall, ACS Nano 2013, 7, 8870-8880.
[14] A. Nagai, N. Koike, H. Kudo, T. Nishikubo, Macromolecules 2007, 40, 8129-8131.
[15] aH. Feil, Y. H. Bae, J. Feijen, S. W. Kim, Macromolecules 1993, 26, 2496-2500; bJ. E. Chung, M. Yokoyama, T. Aoyagi, Y. Sakurai, T. Okano, Journal of Controlled Release 1998, 53, 119-130.
[16] H.-F. Lu, E. D. Targonsky, M. B. Wheeler, Y.-L. Cheng, Biotechnology and Bioengineering 2007, 96, 146-155.
[17] N. Hogg, V. M. Darley-Usmar, M. T. Wilson, S. Moncada, Biochem. J 1992, 281, 419-424.
[18] S. D. Fowler, P. Greenspan, J Histochem Cytochem 1985, 33, 833-836.
[19] L. C. Seaver, J. A. Imlay, J Biol Chem 2004, 279, 48742-48750.
[20] A. J. Convertine, D. S. Benoit, C. L. Duvall, A. S. Hoffman, P. S. Stayton, J Control Release 2009, 133, 221-229.

It will be understood that various details of the presently disclosed subject matter can be changed without departing from the scope of the subject matter disclosed herein. Furthermore, the foregoing description is for the purpose of illustration only, and not for the purpose of limitation.

What is claimed is:

1. A polymer comprising:
a thermally responsive block; and
a hydrophobic block.

2. The polymer of claim 1, wherein the polymer comprises a copolymer.

3. The polymer of claim 2, wherein the copolymer is a terpolymer.

4. The polymer of claim 3, wherein the terpolymer comprises a reactive oxygen species (ROS) degradable ABC triblock.

5. The polymer of claim 4, wherein the terpolymer is thermo-responsive.

6. The polymer of claim 4, wherein the terpolymer comprises poly(propylenesulfide)-block-poly(N,N-dimethylacrylamide)-block-poly(N-isopropylacrylamide) (PPS-b-PDMA-b-PNIPAAM).

7. The polymer of claim 6, wherein the poly(propylenesulfide) block provides antioxidant functionality that reduces cytotoxic oxidative stress for at least one of a cell encapsulated in the polymer, a cell delivered with the polymer, and a local host environment.

8. The polymer of claim 1, wherein the polymer forms a reactive oxygen species-triggered active agent release.

9. The polymer of claim 1, wherein the polymer assembles into stable micelles in aqueous solution.

10. The polymer of claim 9, wherein the micelles include a hydrophobic PPS core.

11. The polymer of claim 10, further comprising an active agent loaded in the PPS core.

12. The polymer of claim 11, wherein the active agent comprises a hydrophobic small molecule drug.

13. The polymer of claim 9, wherein the polymer transitions to a hydrogel at about 37° C.

14. A polymer comprising a thermo-responsive poly(propylenesulfide)-block-poly(N,N-dimethylacrylamide)-block-poly(N-isopropylacrylamide) (PPS-b-PDMA-b-PNIPAAM).

15. A method of forming a polymer, comprising:
anionic polymerization; and
reversible addition-fragmentation chain transfer (RAFT) polymerization.

16. The method of claim 15, wherein the anionic polymerization forms a poly(propylenesulfide)-block of the polymer.

17. The method of claim 15, wherein the RAFT polymerization forms at least one of a poly(N,N-dimethylacrylamide)-block and a poly(N-isopropylacrylamide)-block of the polymer.

18. The method of claim 15, further comprising forming a poly(propylenesulfide)-4-Cyano-4-(ethylsulfanyltiocarbonyl) sulfanylpentanoic acid-RAFT macro-chain transfer agent.

19. The method of claim 15, wherein the polymer is a triblock polymer

20. The method of claim 15, wherein the polymer provides a reactive oxygen species-triggered active agent release.
